(19) United States
(12) Patent Application Publication
(10) Pub. No.: US 2014/0024887 A1
(43) Pub. Date: Jan. 23, 2014

(54) DEVICE FOR HOLDING LIVING TISSUE

(71) Applicant: TERUMO KABUSHIKI KAISHA, Tokyo (JP)

(72) Inventors: Naoki ISHII, Kanagawa (JP); Masakatsu KAWAURA, Kanagawa (JP)

(73) Assignee: TERUMO KABUSHIKI KAISHA, Tokyo (JP)

(21) Appl. No.: 14/039,058

(22) Filed: Sep. 27, 2013

Related U.S. Application Data
(63) Continuation of application No. PCT/JP2012/050221, filed on Jan. 10, 2012.

(30) Foreign Application Priority Data
Mar. 28, 2011 (JP) 2011-070306

(51) Int. Cl. A61B 17/02

(52) U.S. Cl. CPC A61B 17/02
USPC 600/37

(57) ABSTRACT

A device for holding a living tissue including elongated bodies capable of being inserted into a body cavity, and a holder unit provided at a distal end portion of the elongated bodies and exerting an adhesion force whereby the device is able to hold the living tissue by the action of the adhesion force in a liquid containing water in the body cavity.

### CROSS REFERENCES TO RELATED APPLICATIONS

This application is a continuation of International Application No. PCT/JP2012/050221 filed on Jan. 10, 2012, and claims priority to Japanese Application No. 2011-070306 filed on Mar. 28, 2011, the entire content of both of which is incorporated herein by reference.

### TECHNICAL FIELD

This invention generally relates to a device for holding a living tissue and, more particularly, to a device capable of holding an organ in a state where a liquid is infused into a body cavity.

### BACKGROUND DISCUSSION

In recent years, clinical observations and treatments have been made by insertion of an endoscope or the like into a body cavity. Of these, there is a known procedure wherein observation and treatment of a body cavity is performed by infusing air or a physiological saline solution into the body cavity. For instance, there is set forth, in Japanese Patent Laid-open No. 2002-125920, endoscopic observation of the organs inside the abdominal cavity such as the ovary, the back surface of the uterus, the fimbriae of uterine tube and the like, is performed by transvaginally inserting an instrument into the cavity and infusing a physiological saline solution into the cavity.

As a transvaginal treatment, mention are made, for example, of treatments for hydrops folliculi, ovary cyst and the like. The hydrops folliculi is such a clinical state that the bodily secretion is built up in the oviduct cavity, and the ovary cyst is a clinical state where the bodily secretion is built up in the ovary. One method of treatment for these disease states is to transvaginally insert a hollow puncture needle into the abdominal cavity so as to permit the puncture needle to puncture the fallopian tube or ovary, followed by suctioning the internal secretion.

The living body, into which a liquid such as a physiological saline solution or the like is infused, has the possibility that an organ will be free to move by floating in the liquid. If the organ is free to move by floating, endoscopic observation or treatment of such an organ becomes more difficult. However, since the organ is delicate in nature, a procedure of strongly grasping the organ is not favorable.

### SUMMARY

The disclosure herein provides a device for holding a living tissue, which is capable of holding the living tissue without imposing, for acting on the living tissue in the body cavity without a load or force more than necessary to achieve the same.

An exemplary embodiment (i.e., an embodiment disclosed by way of example) of a device for holding a living tissue includes an elongated body capable of being inserted into a living body, and a holder unit provided at a distal end portion of the elongated body and exerting an adhesion force whereby the living tissue is held by the generation of the adhesion force in a liquid medium containing water in the living body.

The device for holding a living tissue configured as described above is able to hold a living tissue in a water-containing liquid medium by the exertion of the adhesion force, so that the living tissue can be held without imposing a load more than necessary and thus, more proper observation or treatment is ensured.

The preferred aspects of the exemplary embodiments of the disclosure are described below.

If the holder unit is configured so as to be deformable or movable by operation at a proximal end side of the elongated body thereof, the living tissue can be held as desired by deformation or movement after insertion of the holder unit in the living body.

When the holder unit has an opening portion to open radially of the elongated body when it is deformed by movement toward the distal end relative to the elongated body, the opening portion ensures holding of the living tissue over a wider region thereof. Thus, the influence on the living tissue can be reduced.

When the holder unit further includes a wire member capable of resilient deformation, the living tissue can be readily held by only adhesion of the wire member.

Moreover, at least one channel is preferably provided inside the elongated body. This allows a device for observation or treatment, such as an endoscope or a puncture needle, to be inserted therethrough.

Moreover, when a hollow or solid needle member capable of moving through the channel is provided, the held living tissue can be readily punctured.

When a pulling wire is provided that is fixed at one end thereof to the distal end portion of the elongated body and is capable of flexing the elongated body by operating at the other end on the proximal end of the elongated body, the elongated body inside the body cavity can be turned around as desired, enabling it to reach at a site or portion that would normally be considered inaccessible and to allow observation or treatment.

Further, the holder unit has an adhesion portion such that a plurality of protrusions are formed and the protrusions are brought into contact with the living tissue to permit the protrusions to be adhered to the living tissue by the Van der Waals' forces, and an adhesion force is shown even in liquid medium. This adhesion force is achieved by a weak pressing force, so that an influence on the living tissue can be reduced and minimized.

A proper adhesion force is ensured in a liquid medium if not less than one per 100 μm² of protrusions are formed having a length of 1 μm to 50 μm and a maximum outer diameter of 5 nm to 10 μm.

When the holder unit is configured to have a protrusion base which is formed protruding from the outer surface of the holder unit and is provided with a protrusion forming face inclined relative to the outer surface, the protrusions are formed on individual protrusion forming faces. In this exemplary configuration, when the inclined protrusion forming faces are removed from the living tissue, the faces come off from one side thereof because of the inclination. This enables the protrusions formed on the protrusion forming faces to be readily withdrawn from the living tissue.

When the holder unit is provided with an adhesion portion having an adhesive material capable of showing an adhesion force in a liquid medium, the living tissue can also be stably held in the liquid medium.

### BRIEF DESCRIPTION OF THE DRAWINGS

The accompanying drawings are included in the specification and form a part of the disclosure here, and are used to disclose aspects and principles of the disclosure here together with the detailed description described below.

FIG. 1 is a plan view showing a living tissue holding device according to a first exemplary embodiment of the disclosure.

FIG. 2 is a sectional view taken along line 2-2 of FIG. 1.

FIG. 3 is a partially enlarged perspective view showing part of an adhesion portion of the living tissue holding device according to the first exemplary embodiment of the disclosure.

FIG. 4 is a sectional view showing protrusions of the living tissue holding device according to the first exemplary embodiment of the disclosure.

FIG. 5 is a sectional view showing a modified example of the protrusions of the living tissue holding device according to the first exemplary embodiment of the disclosure.

FIG. 6 is a plan view showing the living tissue holding device in use according to the first exemplary embodiment of the disclosure.

FIG. 7 is a plan view showing a state where protrusion bases of the living tissue holding device according to the disclosure are in contact with a living tissue;

FIG. 8 is a partially enlarged section view showing adhesion of the protrusions of the living tissue holding device according to the first exemplary embodiment of the disclosure to living tissue.

FIG. 9 is a view showing the protrusion bases of the living tissue holding device of the first exemplary embodiment of the disclosure, which are taken off from the living tissue.

FIG. 10 is a partially enlarged view showing a mold for making the protrusions.

FIG. 11 is a partially enlarged sectional view showing pouring of a material in the mold.

FIG. 12 is a partially enlarged sectional view showing removal of the protrusions from the mold.

FIG. 13 is a partially enlarged sectional view showing another modification example of protrusions.

FIGS. 14A and 14B are, respectively, a view showing a living tissue holding device according to a second exemplary embodiment of the disclosure wherein a holder unit is not opened in FIG. 14A and is opened in FIG. 14B.

FIGS. 15A and 15B are, respectively, a view showing a living tissue holding device according to a third exemplary embodiment of the disclosure wherein a holder unit is not opened in FIG. 15A and is opened in FIG. 15B.

FIGS. 16A and 16B are, respectively, a view showing a living tissue holding device according to a fourth exemplary embodiment of the disclosure wherein a holder unit stands retracted in FIG. 16A and is advanced in FIG. 16B.

FIG. 17 is a view showing the living tissue holding device according to the fourth exemplary embodiment as viewed from a distal end thereof.

FIG. 18 is a view showing how to hold a living tissue with the living tissue holding device according to the fourth exemplary embodiment.

FIG. 19 is a view showing a modifying example of the living tissue holding device according to the fourth exemplary embodiment.

FIG. 20 is a view showing another modifying example of the living tissue holding device according to the fourth exemplary embodiment.

FIGS. 21A and 21B are, respectively a view of a living tissue holding device according to a fifth exemplary embodiment of the disclosure wherein a holder unit thereof stands retracted in FIG. 21A and is advanced in FIG. 21B.

FIG. 22 is a view showing how to hold a living tissue with the living tissue holding device according to the fifth exemplary embodiment.

### DETAILED DESCRIPTION

Reference is made to the exemplary embodiments of the disclosure herein with reference to the accompanying drawings wherein dimensions are just for reference and may differ from actual dimensions for convenience of illustration. It will be noted that like reference numerals are used to indicate like parts or members throughout the drawings.

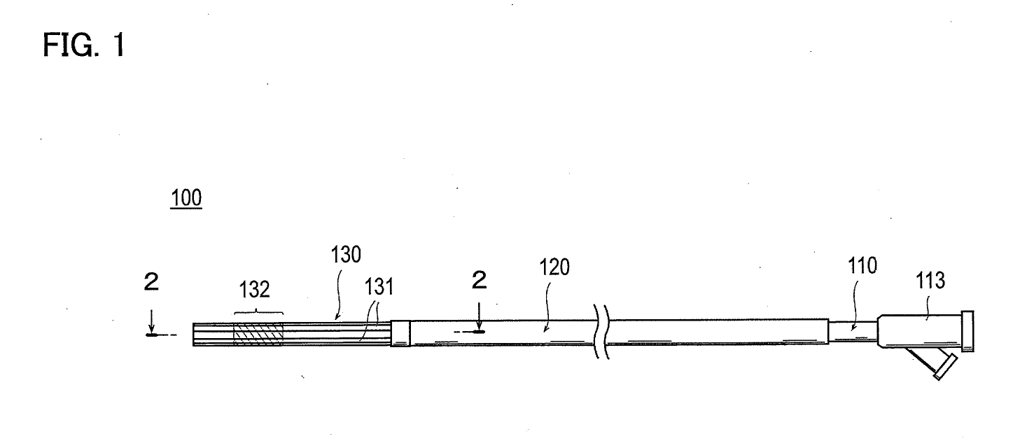

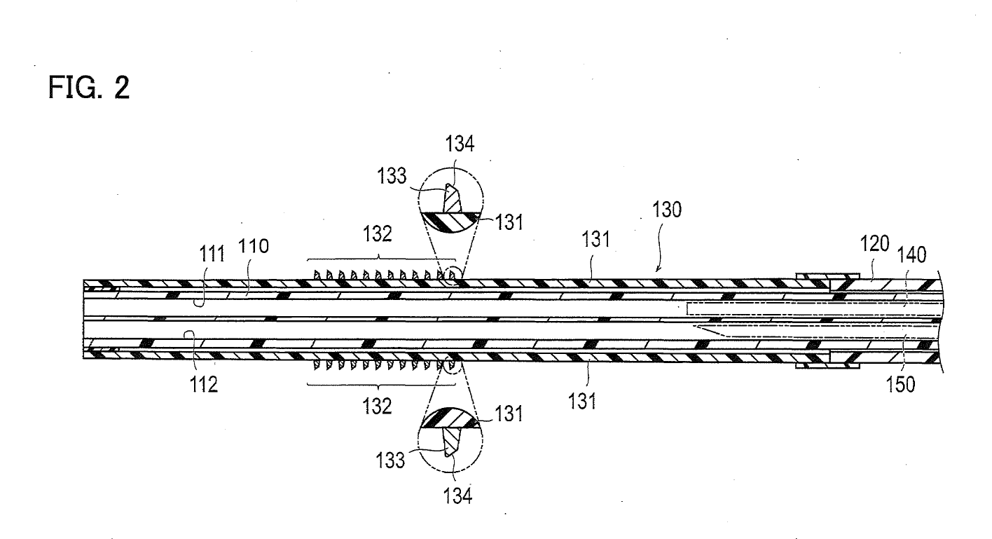

FIGS. 1 and 2 show a living tissue holding device 100 according to a first exemplary embodiment of the disclosure here. This living tissue holding device 100 is used to hold and treat an organ M (living tissue) in the body cavity in a state where a physiological saline solution is infused into the body cavity, for example, as a liquid medium.

The organ M held with the living tissue holding device 100 includes, for example, ovary, fallopian tube or the like. It will be noted that a target to be held is not specifically limited so far as it is composed of a living tissue.

As is particularly shown in FIGS. 1 and 2, the living tissue holding device 100 according to the first exemplary embodiment includes an inner tube (elongated body) 110 having a first channel 111 and a second channel 112 formed therein and an outer tube (elongated body) 120 into which the inner tube 110 is inserted. An endoscope 140 can be inserted into the first channel 111 of the inner tube 110 and a hollow puncture needle 150 (needle member) can be inserted into the second channel 112. A hub 113, which enables the endoscope 140 and the puncture needle 150 to be inserted into the first channel 111 and the second channel 112 while maintaining liquid tightness, is provided at a proximal end of the inner tube 110.

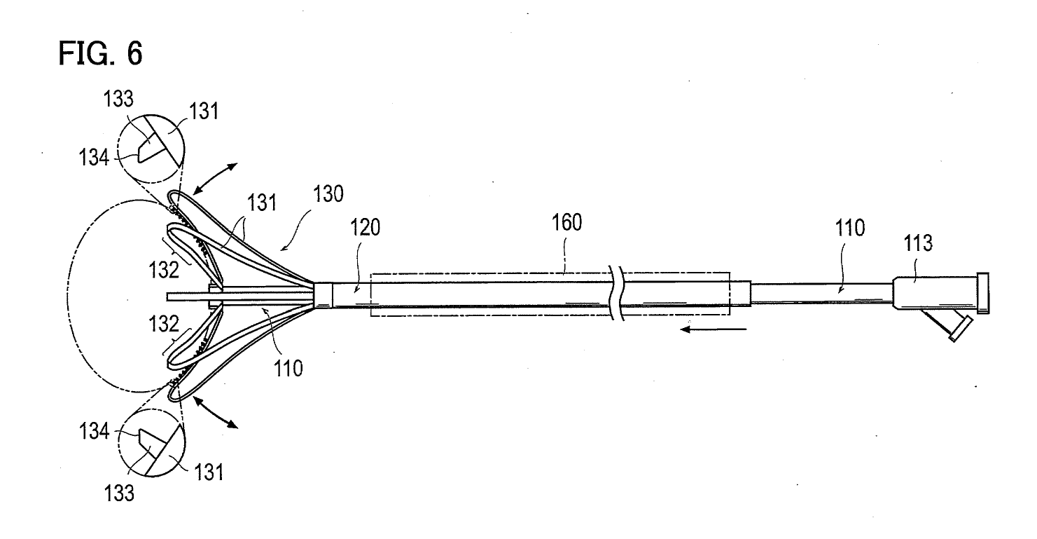

At a distal end of the inner tube 110, there is provided a holder unit 130 brought into contact with the outer surface of the inner tube 110. The holder unit 130 is provided with a plurality (eight in this exemplary embodiment) of sheet-shaped members 131 (opening portions) circumferentially set on the outer surface of the inner tube 110. The sheet-shaped members 131 are bonded at the distal end side thereof with the distal end of the inner tube 110 and at the proximal end side thereof with the distal end of the outer tube 120. The sheet-shaped member 131 becomes thinner from the proximal end toward the distal end and becomes lower in bending rigidity toward the distal end. Accordingly, as shown in FIG. 6, when the outer tube 120 is moved toward the distal end side relative to the inner tube 110, the individual sheet-shaped members 131 are inclined toward the distal end portion, whose rigidity is lower, while curving outwardly. The individual sheet-shaped members 131 are deformed into a funnel shape that is open toward the distal end. It will be noted that a locking mechanism may be separately provided that is capable of fixing the position of the inner tube 110 relative to the outer tube 120 when the outer tube 120 has been moved.

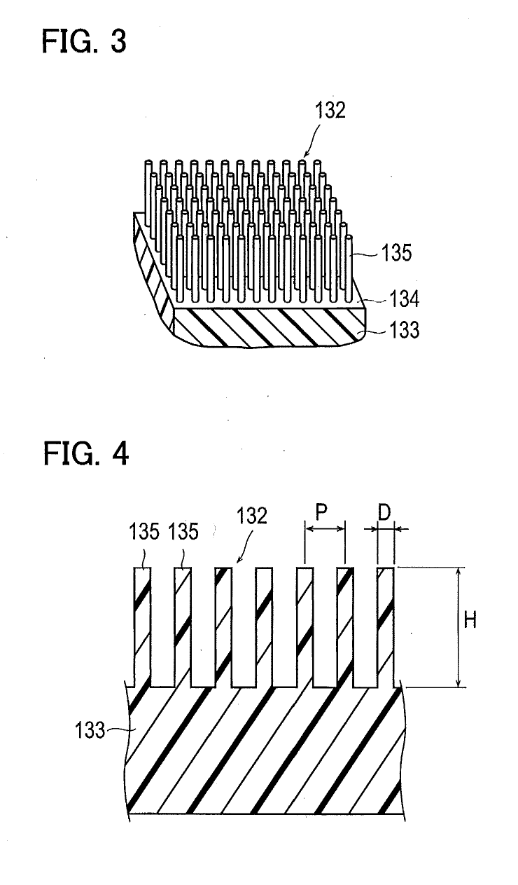

An adhesion portion 132 is provided on the outer surface of the sheet-shaped member 131 at a distal end side ahead of the central portion, i.e. at a radial outer side region of the surface turning to the distal end side in a state of deflection in a funnel shape. The adhesion portion 132 has, as shown in FIG. 2, protrusion bases 133 each projecting from the outer surface of the sheet-shaped member 131. A protrusion forming face 134, which is inclined relative to the outer surface of the sheet-shaped member 131, is formed at an upper portion of the respective protrusion bases 133. The inclination of the protrusion forming face 134 is formed in such a way as to become lower relative to the outer surface of the sheet-shaped member 131 from the distal end side of the device toward the proximal end side. The protrusion forming face 134 is formed with a plurality of fine protrusions 135 on the order of nanometers as shown in FIGS. 3 and 4.

When the adhesion portion 132 with the fine protrusions 135 formed thereon is brought into intimate contact with and pressed against the organ M, a state of attachment or adhesion between the fine protrusions 135 and the organ M can be held by use of the Van der Waals' forces occurring therebetween without separate use of an adhesive. More particularly, when the surface area of the adhesion portion 132 is increased by the provision of a plurality of fine protrusions 135, the Van der Waals' forces result insufficient to maintain adhesion to an object. This adhesion mechanism is shown not only in gas, but also in liquid. With respect to the structure of van der Waals' force-based adhesion, such a fine fibrous structure as seen at the sole of the foot of a gecko is generally known.

As will be described hereinafter, the adhesion portion 132 is formed on the protrusion forming face 134 inclined relative to the outer surface of the sheet-shaped member 131. Accordingly, the coming-off occurs from one side of the protrusion forming face 134 such that if a given force is applied from a predetermined direction, easy coming-off is ensured.

The angle of inclination of the protrusion forming face 134 of the protrusion base 133 relative to the outer surface of the sheet-shaped member 131 can be set appropriately and is not specifically limited. For instance, in the exemplary embodiment, the angle is formed at 5 to 45°, preferably 20 to 30°. The height of the protrusion base 133 is appropriately set and also is not critical, and is, for example, 1 to 50 μm, preferably 10 to 30 μm. The area of one protrusion forming face 134 is appropriately set and is not critical, and is, for example, 1 μm² to 50 μm², preferably 10 μm² to 25 μm². The protrusions 135 are formed at 1 to 10⁶ in number per 100 μm², preferably at 20 to 30 in number per 1 μm².

No specific limitation is placed on the configuration pattern of the protrusion bases 133, which are regularly configured in this exemplary embodiment, but may be configured irregularly.

The protrusions 135 are each formed pillarly (columnarly in this exemplary embodiment). The maximum outer diameter D of the protrusion 135 is 5 nm to 10 μm, preferably 0.1 μm to 0.5 μm. The height H of the protrusion 135 is 1 μm to 500 μm, preferably 10 μm to 50 μm. The pitch P of the protrusions 135 is 0 μm to 1 μm, preferably 0.05 μm to 0.5 μm. It will be noted that the maximum outer diameter means a length of the longest portion along the section perpendicularly intersecting with the protrusion 135 in its extending direction (or protruding direction) and may be used even when the section is not circular.

The protrusions 135 are formed at one or more per 100 μm², preferably not less than 50 in number per 100 μm². If the protrusions 135 have such a shape and dimension as described above, they can show van der Waals' adhesion forces in either gas or liquid.

The configuration pattern of the protrusions 135 is not critical and is regularly arranged in this embodiment, but may be arranged irregularly.

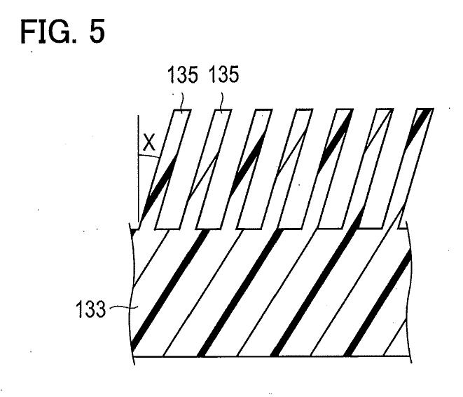

Although each protrusion 135 is formed as vertically extending from the protrusion forming face 134, it may be formed as inclined relative to the protrusion forming face 134 as with the case of another exemplary embodiment shown in FIG. 5. The angle X of inclination can be set at 0 degrees to 60 degrees, preferably 0 degrees to 30 degrees. It will be noted that the direction and angle of inclination may, respectively, differ depending on individual protrusions 135.

The protrusion 135 is not limited to a columnar shape as described before and may be, for example, in pillar form and have a polygonal cross section. The protrusion 135 may not always have the same cross section from the proximal end portion connecting with a substrate 22 to its distal end portion. For instance, the cross section at the distal end portion may be smaller or larger in size than at the proximal end portion.

The protrusion base 133 may be formed integrally with the sheet-shaped member 131, or may be formed by bonding a separate member to the outer surface of the sheet-shaped member 131 by adhesion.

The materials for the sheet-shaped member 131 preferably include those having some flexibility, for which there may be used thermoplastic resins such as general plastics, and thermosetting resins or thermal crosslinking resins such as rubbers. More particularly, mention is made of various types of thermoplastic resins and polymer derivatives thereof including, but not limited to, polyesters such as polyethylene terephthalate, polybutylene terephthalate and the like and polyester elastomers using them as hard segments, polyolefins and polyolefin elastomers such as polyethylene and polypropylene, copolymerized polyolefins making use of polyolefins, polyolefin elastomers and metallocene catalysts, vinyl polymers such as polyvinyl chloride, PVDC, PVDF and the like, polyamides and polyamide elastomers (PAE) including nylons, polyimides, polystyrene, SEBS resins, polyurethanes, polyurethane elastomers, ABS resins, acrylic resins, polyallylate, polycarbonates, polyoxymethylene (POM), polyvinyl alcohol (PVA), fluorine resins (ETFE, PFA and PTFE), ethylene-vinyl acetate saponified products, ethylene-vinyl alcohol copolymers, ethylene vinyl acetate, carboxymethyl cellulose, methylcellulose, cellulose acetate, polyvinyl sulfones, liquid crystal polymers (LCP), polyether sulfones (PES), polyether ether ketone (PEEK), polyphenylene oxide (PPO), polyphenylene sulfide (PPS) and the like, and thermosetting resins or crosslinking resins such as rubbers to be vulcanized, silicone resins such as polydimethylsiloxane (PMDS), polyvinylsilane (PVS) and the like, epoxy resins, two-component reactive polyurethane resins and the like. There may also be used polymer alloys containing any of such thermoplastic resins and thermosetting/crosslinking resins as mentioned above, and resin solutions dissolving resins in liquid may also be used as a molding material.

The inner tube 110 and outer tube 120 should have some rigidity enough to deform the sheet-shaped member 131. For the materials for the inner tube 110 and outer tube 120, there may be used such resin materials as mentioned for the sheet-shaped member 131 and metals such as stainless steels.

The materials for the protrusions 135 are not critical and include, for example those same resin materials as used for the sheet-shaped member 131 and carbon nanotubes formed by bottom up processes.

A procedure of making the resin protrusions 135 is illustrated as representative of one exemplary procedure of making the protrusions 135 on the protrusion base 133.

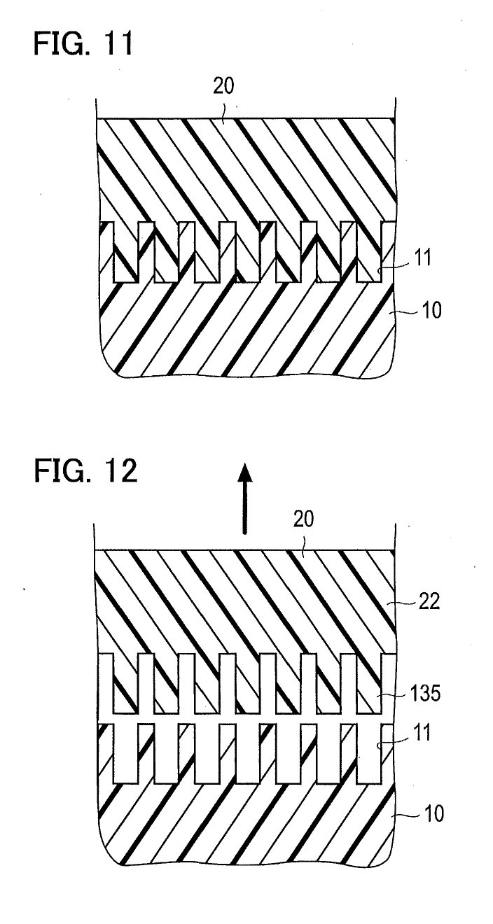

Initially, a fine pore pattern 11 on the order of several hundreds of nanometers is formed in a polymethyl methacrylate resin (PMMA) supported on a silicon wafer according to electron lithography to provide a mold 10 (see FIG. 10). The shape of the fine pattern 11 is determined so as to coincide with the transferred shape of the protrusions 135 of the protrusion forming face 134 which are to be made.

Next, the resin material used as a material for the protrusion 135 is dissolved in a liquid medium at a concentration of 0.001 to 1 wt % thereby providing a sol phase. For the liquid medium, chloroform or the like may be used.

Thereafter, the face of the mold 10 on which the fine pattern 11 has been formed is made horizontal while turning upward, and the sol phase material is poured into the mold 10 as is shown in FIG. 11. In this condition, the material is infiltrated into the fine pattern 11, followed by further pouring the material to a thickness corresponding to a given thickness of the substrate 22. Subsequently, the mold 10 is heated to room temperature to 40° C. and the liquid medium is evaporated to solidify the material. Where the material is thermoplastic in nature, it is thermally melted and poured into the mold 10, followed by cooling for solidification.

After the solidification of the material, the solidified material is removed from the mold 10, thereby obtaining a sheet 20 wherein a plurality of protrusions 135 are formed on the substrate 22. The sheet 20 is then bonded on the protrusion forming face 134 of the sheet-shaped member 131, which may be made in a separate step, thereby providing a configuration of the protrusions 135 formed on the protrusion forming face 134. It will be noted that the protrusion base 133 may be formed integrally with the formation of the protrusions 135 at the same time.

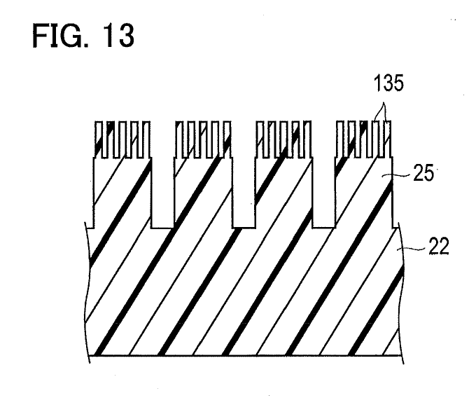

According to the above-described exemplary procedure, a plurality of protrusions 135 may be formed as projecting from individual protrusions 25 formed on the substrate 22, as is particularly shown in FIG. 13. The protrusions 135 may also be formed in conical or pyramidal shape.

It should be noted that for pattern processing on the order of several hundreds of nanometers, there may be used, aside from the above-stated procedure, nanoimprinting, soft lithography, shaping using a fine bit (e.g. a diamond bit) and the like. It is preferred to choose an appropriate procedure depending on the conditions concerning shape, dimension and type of material. If a pyramidal shape is desired, the pattern may be readily formed by forming crisscross grooves using a fine bit.

Explanation of a procedure using the living tissue holding device 100 according to this exemplary embodiment is provided below wherein a method of treating hydrosalpinx (or ovarian cyst) in the abdominal cavity is performed.

Initially, a known trocar wherein an inner needle (not shown) is inserted into a separately provided cylindrical external tube 160 (see FIG. 6) is transvaginally inserted and punctured into Douglas' pouch, thereby permitting it to arrive at the abdominal cavity. Thereafter, the inner needle is withdrawn while leaving the external tube 160, after which a separate water infusion mechanism (not shown) is used to infuse a physiological saline solution into the abdominal cavity via the external tube 160.

Next, the endoscope 140 is inserted into the first channel 111 of the living tissue holding device 100, which is in turn inserted into the external tube 160. At this stage, since the protrusion forming face 134 on which the fine protrusions 135 are formed is inclined, the protrusions 135 are unlikely to be attached to the inner surface of the external tube 160. It will be noted that in order to prevent the protrusions 135 from attachment to the inner surface of the external tube 160, a low friction member such as a fluorine resin may be coated thereon. While observing the inside of the abdominal cavity with the endoscope 140, the living tissue holding device 100 is pushed ahead until it arrives at an intended position. After the arrival at the fallopian tube that is an intended organ M, fine adjustment is made such that a site to be punctured is located ahead of the second channel 112, under which the outer tube 120 is moved toward the distal end side relative to the inner tube 110. In this way, as shown in FIG. 6, the sheet-shaped member 131 is deformed in a funnel fashion.

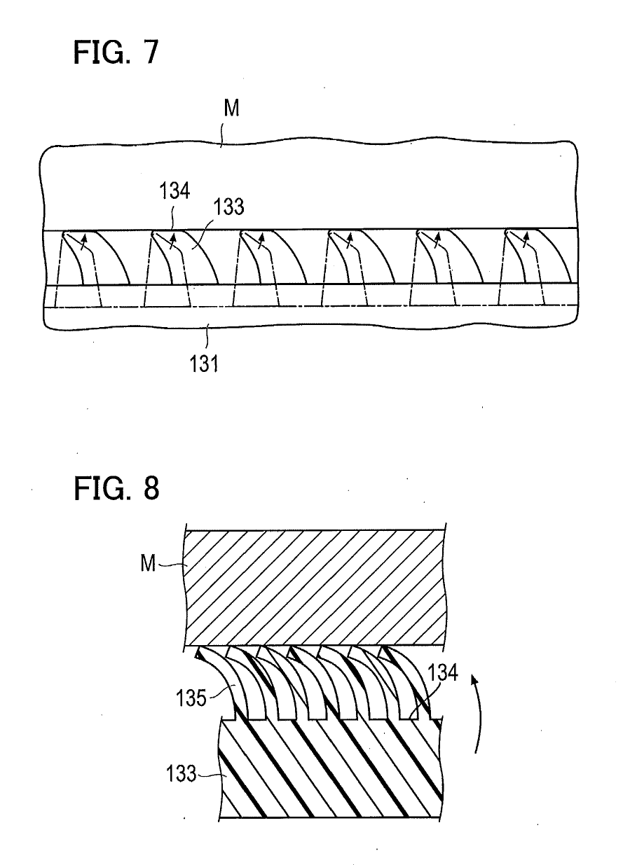

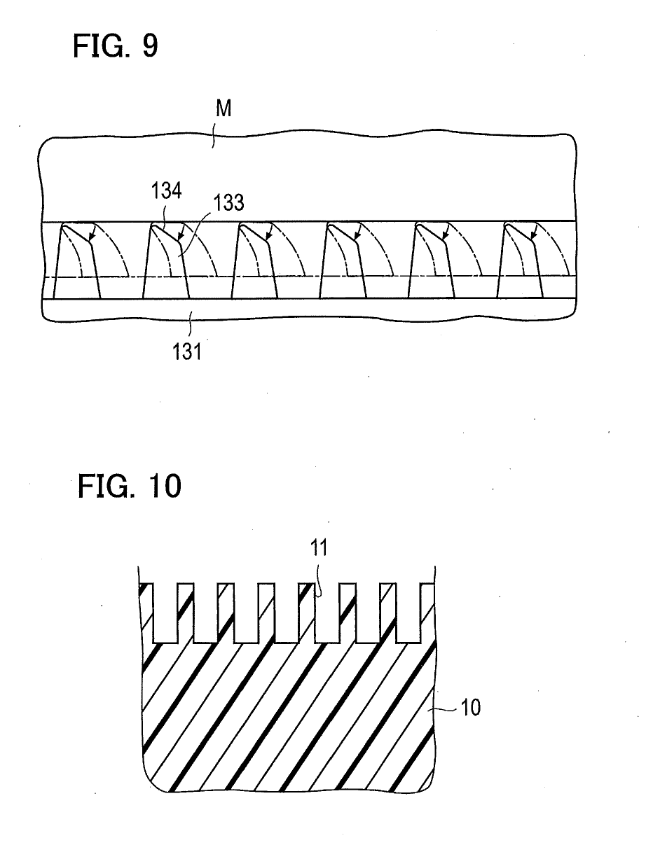

During the course of the funnel-shaped deformation, the adhesion portion 132 of the sheet-shaped member 131 is forced against the fallopian tube. This permits the adhesion portion 132 to gradually come in contact with the fallopian tube from the distal end side thereof (i.e. a side connecting to the inner tube 110) while the sheet-shaped member 131 is expanding outwardly and deforming toward the distal end side. Finally, the sheet-shaped member 131 holds the fallopian tube by wrapping therearound. At this time, as shown in FIG. 7, the protrusion base 133 deforms so that the protrusion forming faces 134 that are formed at an incline come in contact with the organ M. Then, as shown in FIG. 8, since the adhesion portion 132 has a plurality of protrusions 135, the organ M is held by adhesion to the adhesion portion 132 by the action of the Van der Waals' forces. It will be noted that the pressing force may be made smaller than a press force such as for puncture, so that adhesion even to a floating organ M in liquid is possible. Moreover, pressing is made so as to wrap the organ M and thus, a pressing force is likely to be developed against the floating organ M. Additionally, the sheet-shaped member 131 is deformed in funnel fashion and adhered to the organ M so as to wrap therearound. Hence, the organ M can be appropriately held without imposing a load more than necessary thereto.

After holding the organ M with the holder unit 130, a puncture needle 150 is inserted into the second channel 112 of the living tissue holding device 100, and is advanced and punctured into the organ M while observing a puncture position by means of the endoscope 140. In this instance, the organ M is held with the holder unit 130 and, more particularly, is held with the holder unit 130 at a plurality of positions about the outer periphery of the puncture needle 150. Thus, puncture at a precise position is ensured, thereby improving safety.

After the puncture at the intended position, the bodily secretion built up in the organ M is discharged by suction via the hollow puncture needle 150. Thereafter, the puncture needle 150 is retracted, followed by removal from the organ M. The outer tube 120 is also moved back relative to the inner tube 110, and the sheet-shaped member 131 which was deformed into a funnel shape is returned to its original shape. When returned to the original shape, the sheet-shaped member 131 in open condition is gradually stepped away from the organ M from the radial outer side (i.e. a side connecting with the outer tube 120) of the adhesion portion 132 of the open sheet-shaped member 131. Since the lower side of the protrusion base 133 is located at the radial outer side, the adhesion portion is separated off from the lower side of the protrusion base 133. Accordingly, the protrusion forming faces 134 of the protrusion base 133 are removed from one direction and thus, the protrusions 135 can be readily removed without imposing a load more than necessary to the organ M. In this way, the adhesion portion 132 can be adhered to and retracted from the organ M without imposing a load more than necessary, so that if the adhesion portion 132 cannot be held at a position intended for the adhesion, it becomes possible to retract from and then hold it again without treatment (puncture).

Thereafter, the living tissue holding device 100 is withdrawn from the external tube 160 and the physiological saline solution in the abdominal cavity is discharged via the external tube 160, followed by removal of the external tube 160 and thereby completing the procedure.

According to this exemplary embodiment, the holder unit 130 provided at the distal end portions of the outer tube 120 and the inner tube 110 to be inserted into the body cavity is formed with the protrusions 135 to be adhered to the organ M by the generation of van der Waals' forces, the organ M can be held with this holder unit 130 in a liquid containing water in the body cavity. Thus, the held organ M can be observed, treated or moved. Moreover, since the Van der Waals' forces are used for the holding, great holding power can be developed under a small pressing force, thereby ensuring a reduced influence on the organ M.

The holder unit 130 has the sheet-shaped member 131 which can be deformed or moved by operations at the proximal end sides of the outer tube 120 and inner tube 110 and thus, after insertion into the body cavity, it can be deformed or moved to hold the organ M.

The holder unit 130 also has an opening portion which is able to deform so as to open in radial directions of the outer tube 120 and inner tube 110 and can hold the organ M by deformation or movement after insertion into the body cavity.

Since the first channel 111 and second channel 112 are formed inside the inner tube 110, a device for observation or treatment, such as the endoscope 140 or puncture needle 150, can be inserted thereinto.

The hollow puncture needle 150 capable of moving inside the second channel 112 is provided, so that the held organ M can be punctured.

A living tissue holding device 200 according to the second exemplary embodiment of the disclosure differs from the living tissue holding device 100 according to the first exemplary embodiment only in the structure of a holder unit 230.

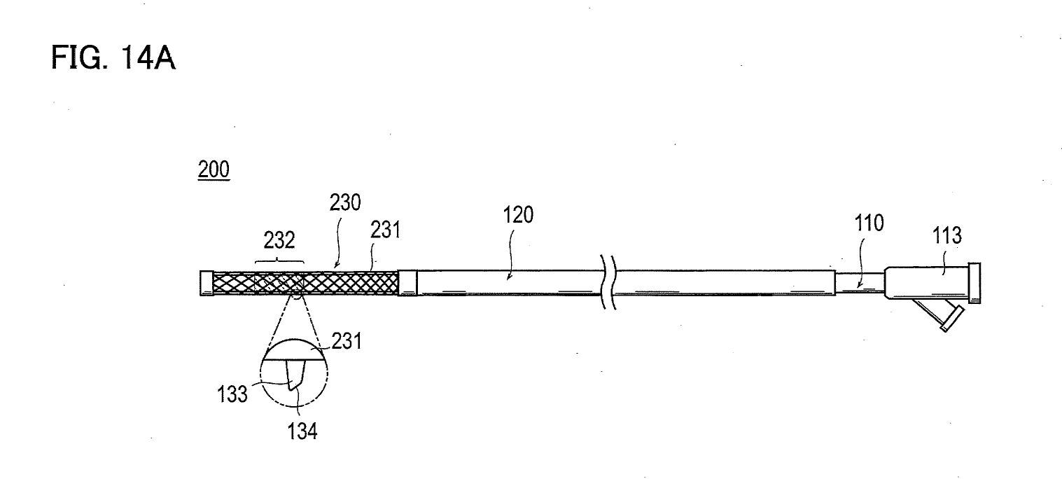

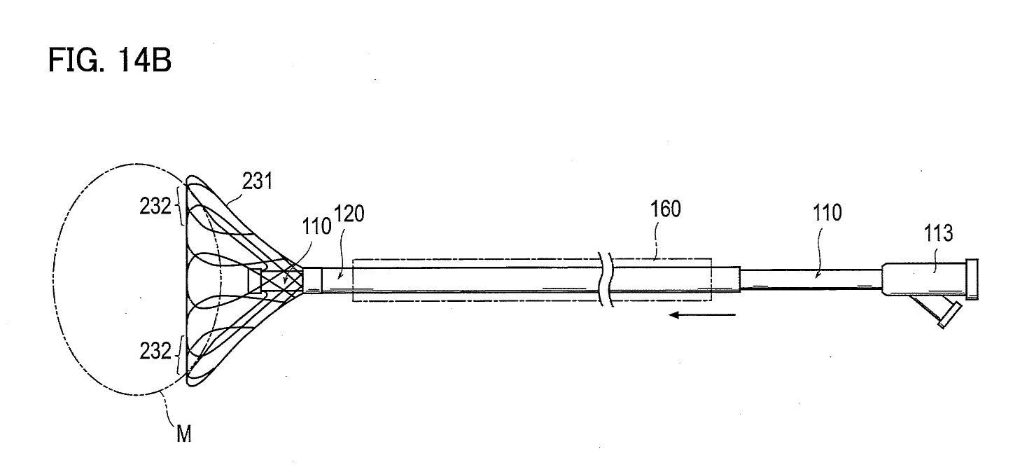

The holder unit 230 of the living tissue holding device 200 according to a second exemplary embodiment includes, as shown in FIGS. 14A and 14B, a mesh-like member 231 which is reticulated by the use of a plurality of wires. The mesh-like member 231 has a structure that becomes coarser at the distal end side than at the proximal end side and has a bending rigidity which becomes lower toward the distal end side. Accordingly, as shown in FIG. 14B, when the outer tube 120 is moved, relative to the inner tube 110, toward the distal end side, the respective wires are inclined toward the distal end side whose rigidity is low while flexing outwardly and deformed into a funnel shape opening toward the distal end side.

The wire materials for the mesh-like member 231 are not critical so far as they are resiliently deformable and include, for example, stainless steels, super elastic metals (e.g. Ni—Ti alloys) and the like.

The mesh-like member 231 has on the outer surface thereof an adhesion portion 232 having a plurality of fine protrusions 135 on the order of nanometers at a distal end side ahead of the central portion, i.e. a portion of the radial outer side relative to the surface facing to the distal end side of the device in a state of being opened in a funnel fashion as is particularly shown in FIG. 14B.

The protrusions 135 are formed on the protrusion forming face 134 of the protrusion base 133 located as projected from the mesh-like member 231. In order to increase the area of the adhesion portion 232, a film member capable of expansion along with the mesh-like member 231 may be provided on the reticulate member 231 so as to form the protrusion base 133 and the protrusions 135 on the film member. The film member is designed to be expanded elastically or from a folded state thereof, by which it becomes expandable integrally with the reticulate member 231. The materials for the film member include silicone rubber materials such as polydimethylsiloxane (PDMS) and the like as an elastically expandable one and nonwoven fabrics woven from nanofibers or porous films such as of polytetrafluoroethylene (PTFE) and the like as an expandable one from a folded state.

When using the living tissue holding device 200 according to the second embodiment, the organ M can be held in a liquid containing water by the generation of van der Waals' forces. Thus, observation, treatment or movement can be performed while holding the organ M without imposing a load more than necessary.

A living tissue holding device 300 according to a third exemplary embodiment of the disclosure is now described.

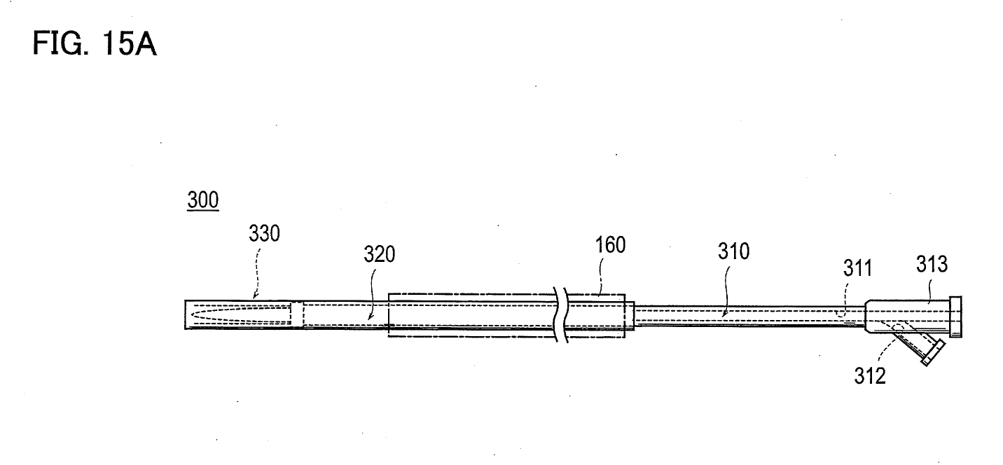

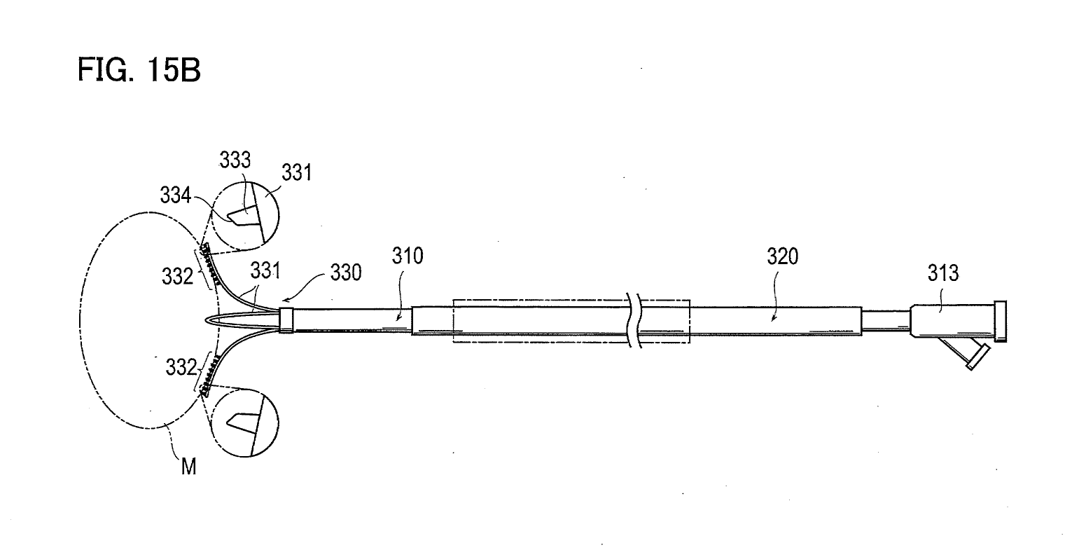

The living tissue holding device 300 of the third exemplary embodiment includes, as shown in FIGS. 15A and 15B, an inner tube 310 wherein a first channel 311 and a second channel 312 are formed, and an outer tube 320 into which the inner tube 310 is inserted. An endoscope 140 is insertable into the first channel 311 of the inner tube 310 and a hollow puncture needle 150 is insertable into the second channel 312. At the proximal end side of the inner tube 310, there is provided a hub 313 through which the endoscope 140 and the puncture needle 150 are insertable.

A holder unit 330 that is able to expand in radially outward directions in a natural state is provided at the distal end portion of the inner tube 310. The holder unit 330 is provided with a ring-shaped member 331 circularly formed of a resiliently deforming wire, and a plurality of ring-shaped members 331 are provided (four in this exemplary embodiment) in juxtaposition along the circumferential direction of the inner tube 310.

When the outer tube 320 is moved toward the distal end relative to the inner tube 310 so as to permit the outer tube 320 to cover the ring-shaped member 331 therewith, the ring-shaped member 331 is resiliently deformed and fits inside the outer tube 320. On the other hand, when the outer tube 320 is moved toward the proximal end, the ring-shaped member 331 resiliently expands. It will be noted that there may be provided a locking mechanism capable of fixing the position between the inner tube 310 and the outer tube 320 in a state wherein the outer tube 320 has been moved.

An adhesion portion 332 is formed at a radially outward side of the surface facing to the distal end side in a case where the ring-shaped member 331 is expanded. At the adhesion portion 332, there are formed protrusion bases 333 projecting from the outer surface of the ring-shaped member. A protrusion forming face 334 inclined relative to the outer surface of the ring-shaped member 331 is formed upwardly of each protrusion base 333. The protrusion forming face 334 is formed as inclined in such a way as to become lower than the outer surface of the ring-shaped member 331 from the distal end side toward the proximal end side. The protrusion forming face 334 is formed thereon with a plurality of fine protrusions 135 on the order of nanometers.

The living tissue holding device 300 according to the third exemplary embodiment is inserted into the body cavity in such a state that the holder unit 330 is covered with the outer tube 320. For holding the organ M, the outer tube 320 is moved toward the proximal end relative to the inner tube 310 to permit the ring-shaped member 331 to be restored to an expanded (natural) shape. The adhesion portion 332 of the ring-shaped member 331 is pressed against the organ M, whereupon the adhesion portion 332 having the protrusions 135 is adhered to the organ M. In so doing, the organ M is held with the holder unit 330. Thereafter, the puncture needle 150 is inserted into the second channel 312 of the living tissue holding device 300 and is advanced and punctured into the organ M while confirming a puncture position with the endoscope 140. Since the organ M is held with the holder unit 330 and holding at a plurality of positions along the circumference of the puncture needle 150 is ensured, the organ M can be accurately held and punctured at a correct site, thereby improving safety.

After the puncture, the bodily secretion built up in the organ M is suctioned via the hollow puncture needle 150 and discharged. Thereafter, the puncture needle 150 is retracted and pulled out from the organ M. The living tissue holding device 300 is retracted as a whole toward the proximal end side. Since the protrusion forming face 334 is formed as inclined in such a way as to become lower relative to the outer surface of the ring-shaped member 331 from the distal end toward the proximal end, it comes off from one end thereof. Thus, the coming-off becomes possible without imposing a load more than necessary to the organ M. Next, the outer tube 320 is moved toward the distal end relative to the inner tube 310 to place the ring-shaped member 331 in the outer tube 320. Subsequently, the living tissue holding device 300 is withdrawn from the external tube 160 and the physiological saline solution in the abdominal cavity is discharged through the external tube 160, followed by removal of the external tube 160 and completion of the procedure.

According to the third exemplary embodiment, the organ M can also be held with the ring-shaped member 331 provided at the holder unit 330 without imposing a load more than necessary to the organ M thereby enabling observation, treatment or movement thereof.

A living tissue holding device 400 according to a fourth exemplary embodiment of the disclosure is now described.

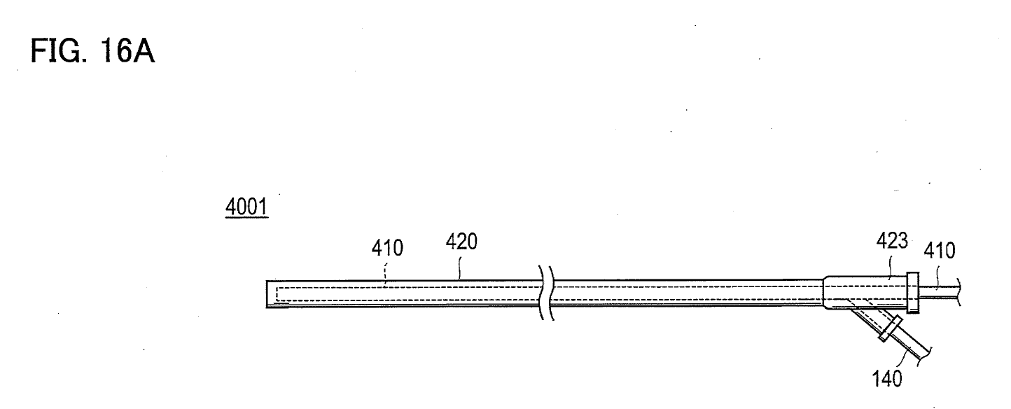

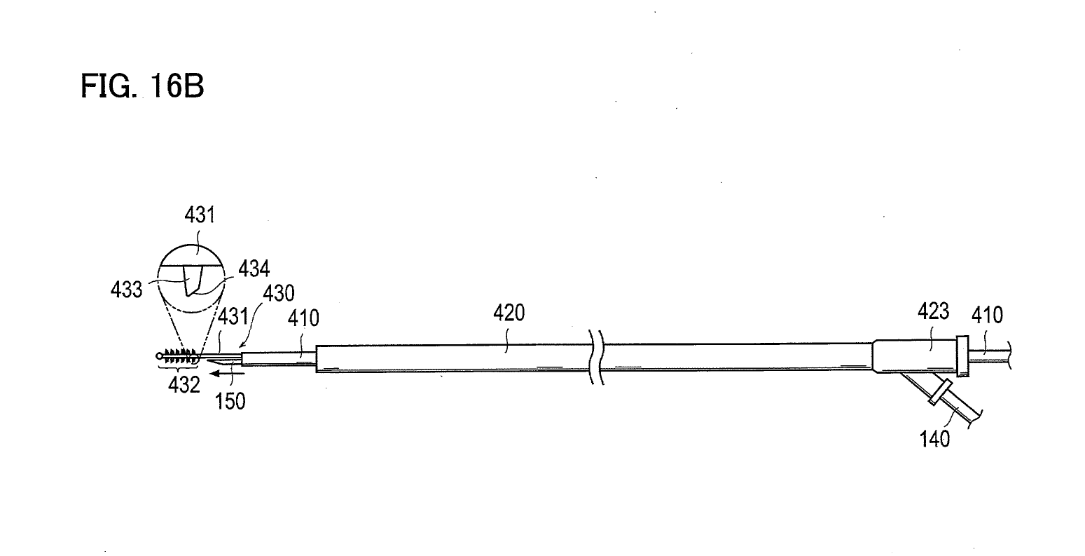

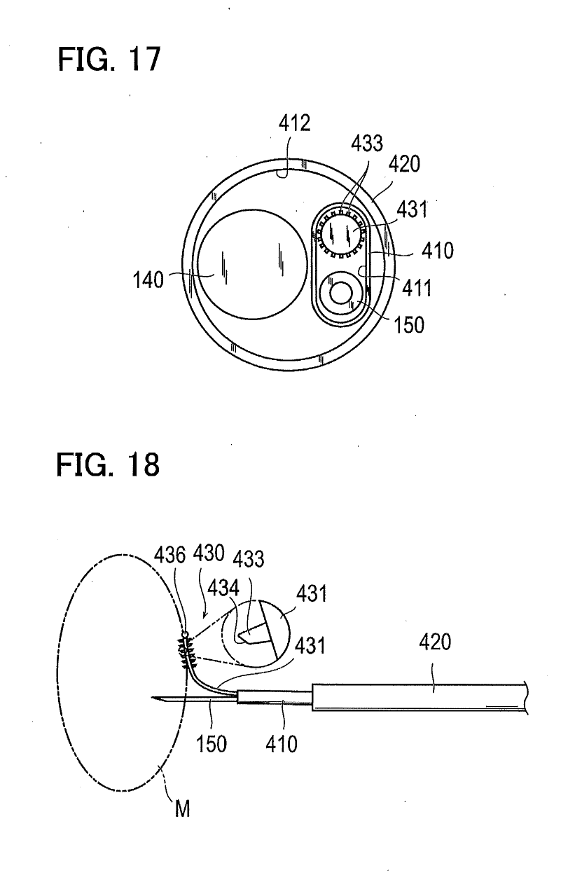

A living tissue holding device 400 of the fourth exemplary embodiment includes, as shown in FIGS. 16A, 16B and 17, an inner tube 410 wherein a first channel 411 is formed, and an outer tube 420 forming a second channel 412 into which the inner tube 410 and an endoscope 140 are to be inserted. An elongated holder unit 430 and a hollow puncture needle 150 are insertable into the first channel 411 of the inner tube 410 in a liquid tight manner. A hub 423, into which the endoscope 140 and the inner tube 410 are insertable, is provided at the proximal end of the outer tube 420.

The holder unit 430 has a wire member 431 formed of a resiliently deformable wire and a spherical body 436 provided at a distal end of the wire member 431 so as to reduce an influence on living body. An adhesion portion 432 is formed on the outer circumferential surface at the distal end of the wire member 431. The adhesion portion 432 is formed with protrusion bases 433 each projecting from the outer surface of the wire member 431. At an upper portion of the respective protrusion bases 433, there is formed a protrusion forming face 434 that is inclined with respect to the outer surface of the wire member 431. The protrusion forming face 434 is formed as inclined to become lower relative to the outer surface of the wire member 431 from the distal end side toward the proximal end side. The protrusion forming face 434 has formed thereon a plurality of fine protrusions 135 on the order of nanometers.

The living tissue holding device 400 is inserted into the living cavity in such a state that the puncture needle 150 and the holder unit 430 are accommodated in the inner tube 410 and the endoscope 140 and inner tube 410 are set in the outer tube 420. For holding the organ M, the inner tube 410 is advanced from the outer tube 420 toward the distal end side while observing with the endoscope 140. Subsequently, as shown in FIG. 18, the wire member 431 of the holder unit 430 is advanced from the inner tube 410 and pressed against the organ M. At this moment, the wire member 431 contacts the organ M from its distal end side and deforms along the living body while being curved, under which the adhesion portion 432 having the protrusions 135 on each protrusion forming face 434 is adhered to the organ M. In this way, the organ M is held with the holder unit 430. Thereafter, the puncture needle 150 is inserted into the first channel 411 of the living tissue holding device 400 and is advanced for puncturing the organ M while confirming a puncture position with the endoscope 140. Since the organ M is held with the holder unit 430, it is possible to make a puncture in correct position, thereby improving safety.

Subsequently, the bodily secretion stored in the organ M is suctioned and discharged through the hollow puncture needle 150, followed by retracting the puncture needle 150 and withdrawing it from the organ M. The holder unit 430 or the living tissue holding device 400 as a whole is retracted and withdrawn from the organ M. Since the protrusion forming face 434 is formed at an inclination in such a way as to become lower relative to the outer surface of the wire member 431 from the distal end side toward the proximal end, the adhesion portion 432 comes off from one side (the proximal end) and can thus be separated without imposing a load more than necessary to the organ M. Thereafter, the holder unit 430 is set in the inner tube 410 and the inner tube 410 is in turn set in the outer tube 420, after which the living tissue holding device 400 is withdrawn from the external tube 160 and the physiological saline solution in the cavity is discharged through the external tube 160, followed by withdrawal of the external tube 160 and completion of the procedure.

According to the fourth exemplary embodiment, since the living tissue can be readily held by only pressing the wire member 431 thereagainst, workability is excellent. The organ M is held with the wire member 431 without imposing a load more than necessary to the organ M, thus enabling observation, treatment or movement thereof.

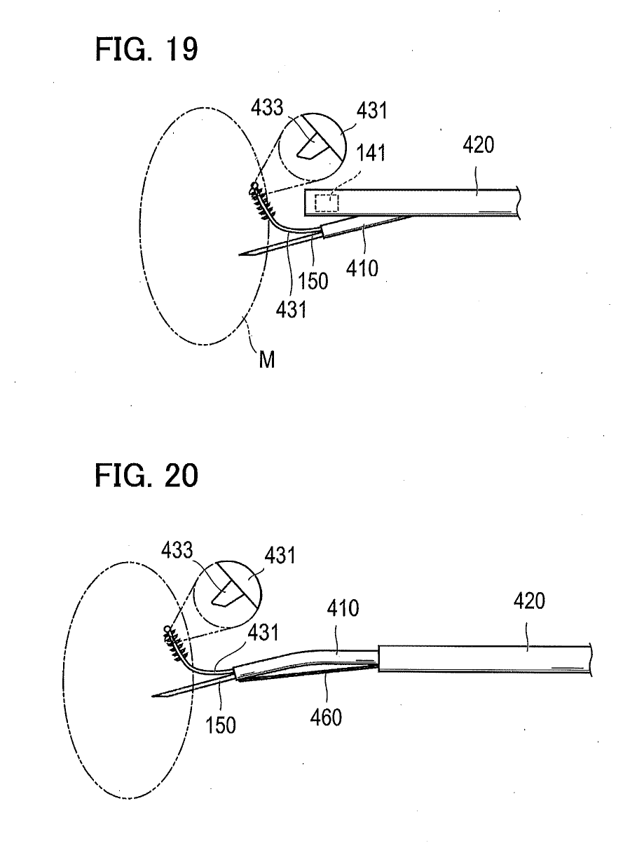

It will be noted that FIG. 19 is a modification of the fourth exemplary embodiment wherein the inner tube 410 is advanced from an opening provided at the side of the outer tube 420. This allows the inner tube 410 to be advanced while avoiding the hindrance of the endoscope 140 which is provided with a solid state pickup device 141 and thus becomes large in size at the distal end portion. As a result, the outer tube 420 can be designed thinner to reduce a load to living body.

FIG. 20 is another modification of the fourth exemplary embodiment wherein a pulling wire 460 is provided between the inner tube 410 and the outer tube 420 and is configured to be fixed at the distal end portion of the inner tube 410. This enables the pulling wire 460 to be handled with one's fingers, so that the inner tube 410 can be bent so as to turn the endoscope 140 and/or the puncture needle 150 around as desired. Thus, it becomes possible to arrive at a site that would normally be considered inaccessible and allow observation or treatment. It will be noted that the pulling wire 460 may be extended by a given length from the distal end of the fixed inner tube 410 toward the proximal end and introduced into the inside of the inner tube 410 from a hole (not shown) formed at the side surface of the inner tube 410.

A living tissue holding device 500 according to a fifth exemplary embodiment of the disclosure is now described.

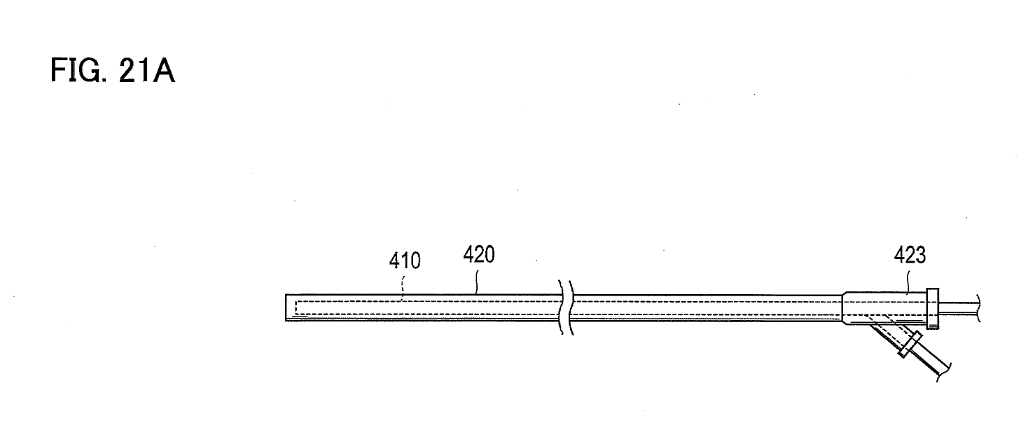

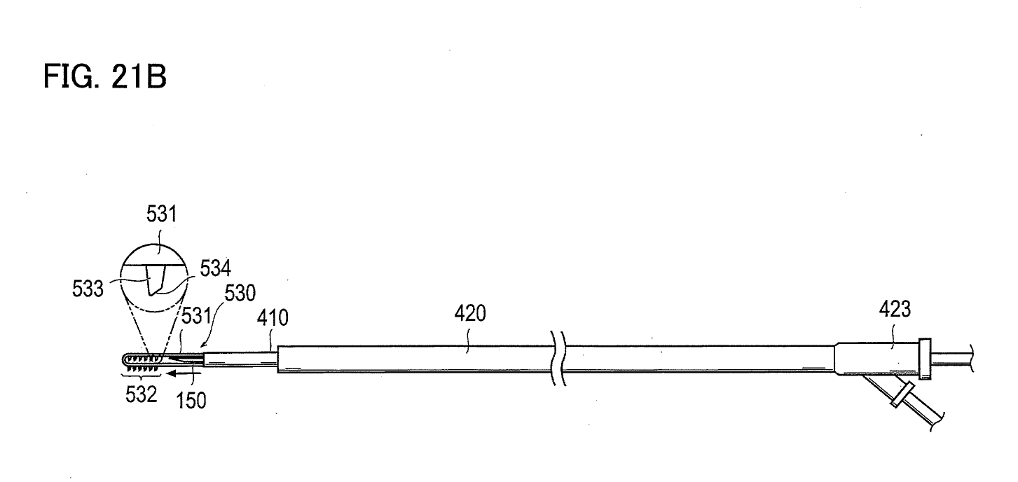

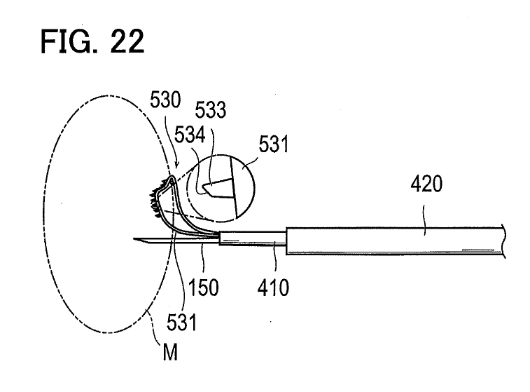

The living tissue holding device 500 according to the fifth exemplary embodiment differs from that of the fourth embodiment in that a wire member 531 of a holder unit 530 is formed as a loop, as shown in FIGS. 21A, 21B and 22. An adhesion portion 532 is formed on the outer surface of the wire member 531 at a distal end thereof. The adhesion portion 532 is formed thereon with protrusion bases 533 each projecting from the outer surface of the wire member 531 and each base has, at an upper portion thereof, a protrusion forming face 534 inclined relative to the outer surface of the wire member 531. The protrusion forming face 534 is inclined in such a way as to become lower relative to the outer surface of the wire member 531 from the distal end side toward the proximal end. A plurality of fine protrusions 135 on the order of nanometers are formed on the protrusion forming face 534.

When using the living tissue holding device 500 of the fifth exemplary embodiment, the wire member 531 of the holder unit 530 is advanced from the inner tube 410 and the adhesion portion 532 of the wire member 531 is pressed against the organ M to permit adhesion of the protrusions 135 as is particularly shown in FIG. 22. In so doing, the organ M can be held without imposing a load more than necessary to the organ M, thus enabling observation, treatment or movement.

The disclosure should not be construed as limited to those exemplary embodiments stated herein above and various modifications and alterations may be possible without departing from the spirit of the invention. For example, the endoscope 140 and the puncture needle 150 may not always be provided, and the channels into which the endoscope 140 or the puncture needle 150 are to be inserted may not always be provided.

Furthermore, the adhesion mechanism is not limited to those of adhesion based on van der Waals' forces by use of the fine protrusions 135. For instance, a pressure-sensitive adhesive (adhesive) capable of showing a tack force (or adhesion force) in a liquid containing water may be applied onto the adhesion portion. Examples of such a pressure-sensitive adhesive (adhesive) include 3,4-dihydroxy-L-phenylalanine (dopamine, DOPA) which is an adhesive peptide having a catechol group and derivatives, polymers and copolymers thereof. Additionally, polysaccharides such as dextran, dextrin and derivative thereof may also be used.

The detailed description above describes a device for holding a living tissue disclosed by way of example. The disclosure here is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A device for holding a living tissue comprising:
at least one elongated body capable of being inserted into a body cavity, and
a holder unit provided at a distal end portion of said at least one elongated body for exerting an adhesion force on the living tissue, whereby said device is able to hold the living tissue by the generation of the adhesion force in a liquid containing water in the body cavity.

2. The device for holding a living tissue according to claim 1, wherein said holder unit is deformable or movable by operations at a proximal end of said at least one elongated body.

3. The device for holding a living tissue according to claim 1, wherein said holder unit has an opening portion which is deformed by movement toward a distal end relative to said at least one elongated body so as to open in radial directions of said elongated body.

4. The device for holding a living tissue according to claim 1, wherein said holder unit has a resiliently deformable wire member.

5. The device for holding a living tissue according to claim 1, wherein said at least one elongated body has at least one channel in the inside thereof.

6. The device for holding a living tissue according to claim 5, further comprising:
a hollow or solid needle member capable of moving through said at least one channel.

7. The device for holding a living tissue according to claim 1, further comprising:
a pulling wire disposed with one end fixed at the distal end portion of said elongated body, wherein when said pulling wire is operated at the other end on the proximal end of said elongated body, said elongated body is turned around.

8. The device for holding a living tissue according to claim 1, wherein said holder unit further includes an adhesion portion having a plurality of protrusions formed thereon and, when said protrusions contact the living tissue, said adhesion portion is adhered to the living body by the generation of van der Waals' forces.

9. The device for holding a living tissue according to claim 8, wherein said protrusions are formed at a density of not less than one per 100 μm² and have a length of 1 μm to 50 μm and a maximum outer diameter of 5 nm to 10 μm.

10. The device for holding a living tissue according to claim 8, wherein said holder unit has protrusion bases each projecting from the outer surface of said holder unit and provided with a protrusion forming face inclined relative to said outer surface, said protrusions being formed on said protrusion forming face.

11. The device for holding a living tissue according to claim 1, wherein said holder unit has an adhesion portion having an adhesive material capable of exhibiting adhesion on said living body in the liquid.

12. A device for holding a living tissue comprising:
an outer tube;
an inner tube having a first channel and a second channel formed therein, said inner tube being disposed within said outer tube;
a holder unit disposed at a distal end of the inner tube, the holder unit including a plurality of members circumferentially disposed about an outer surface of the inner tube;
wherein the members are bonded at a distal end thereof with a distal end of the inner tube and at a proximal end thereof with a distal end of the outer tube.

13. The device for holding a living tissue according to claim 12, wherein, when the outer tube is moved distally relative to the inner tube, the members become inclined toward the distal end of the inner tube.

14. The device for holding a living tissue according to claim 13, wherein the members are deformed into a funnel shape that is facing open towards the distal end of the inner tube.

15. The device for holding a living tissue according to claim 14, wherein the members include an adhesion portion having a plurality of protrusions formed thereon and, when said protrusions contact the living tissue, said adhesion portion is adhered to the living body by the generation of van der Waals' forces.

16. The device for holding a living tissue according to claim 15, wherein said protrusions are formed at a density of not less than one per 100 μm² and have a length of 1 μm to 50 μm and a maximum outer diameter of 5 nm to 10 μm.

17. The device for holding a living tissue according to claim 15, wherein said holder unit has protrusion bases each projecting from an outer surface of said holder unit and provided with a protrusion forming face inclined relative to said outer surface, said protrusions being formed on said protrusion forming face.

18. The device for holding a living tissue according to claim 12, wherein the members include an adhesion portion, said adhesion portion having an adhesive material capable of exerting adhesion on said living body in the liquid.
